(19) United States
(12) Patent Application Publication
(10) Pub. No.: US 2015/0127077 A1
(43) Pub. Date: May 7, 2015

(54) Thermal Stimulation Probe And Method

(71) Applicants: Hanan Ben Asher, Haifa (IL); Amir Haiman, Karkur (IL)

(72) Inventors: Hanan Ben Asher, Haifa (IL); Amir Haiman, Karkur (IL)

(73) Assignee: MEDOC ADVANCED MEDICAL SYSYTEMS, LTD., Ramat Yishai (IL)

(21) Appl. No.: 14/399,488

(22) PCT Filed: May 9, 2013
(86) PCT No.: PCT/IL13/50405

§ 371 Date: Nov. 6, 2014

Related U.S. Application Data
(60) Provisional application No. 61/644,446, filed on May 9, 2012.

(51) Int. Cl. A61F 7/00

(52) U.S. Cl. CPC A61F 7/007; A61F 2007/0075
USPC 607/96

(57) ABSTRACT

A portable device for thermal stimulation of tissue of a patient comprising a heat control element having a proximal side and a distal side wherein a temperature difference can be generated between the proximal side and the distal side, and wherein the proximal side is capable of contacting the tissue. A fan capable of exchanging heat with surrounding air and a heat sink coupled to the distal side are provided. At least one resilient element connected between the heat sink and the fan is also provided, for supporting the fan so as to prevent direct contact between the heat sink and the fan. Furthermore at least one temperature sensor and circuitry are provided, wherein the circuitry activates the heat control element using the at least one temperature sensor, so as to achieve a desired temperature stimulation of the tissue at a rate of substantially 1 degree Celsius per second.

### FIELD OF THE INVENTION

The present invention relates to an improved apparatus, system and method for thermal stimulation of tissue.

### BACKGROUND OF THE INVENTION

The pain mediating system in a human consists of two kinds of afferent fibers: A-delta and C-fibers. These afferent pain fibers are characterized by different physiological parameters, for example, conduction velocity (5-30 m/s for A-delta fibers and 0.5-2 m/s for C-fibers). These two fiber types project to different parts of the dorsal horn of the spinal cord. In addition, stimulation of each kind of nociceptors evokes different type of sensation: A-delta fibers mediate first (sharp, pin-prick) pain sensation; C-fibers mediate the sensation of second pain usually perceived as burning sensation.

Dysfunction of pain- and sensory-mediated systems often accompanies various neurological disorders as well as other pain syndromes of unknown etiology. Therefore, selective activation and identification of the response may offer very significant opportunity for proper diagnostic and treatment in pain patients. A typical tool for the evaluation of A-delta functioning is radiant heat laser stimuli that evoke pin-prick sensation (e.g. user response) and well defined potential on EEG recording. However, selective activating with subsequent recording for the evaluation of C-fibers activity is apparently more difficult. Some existed methods for the selective C-fibers activation are based on laser stimulation following the ischemic block of A-delta fibers; applying laser stimuli on very tiny cutaneous surface areas (d=0.5 mm) using special lens; or by stimulating skin surface through special filters. These methods, however, have not found widespread clinical use, possibly due to their complexity and/or poor sensation generation quality.

Peltier elements have been used for heat and/or cold stimulating a body portion for evaluating nervous sensitivity, for example, as described in U.S. Pat. No. 6,741,895 (Gafni E. et al, “Vaginal Probe and Method”) where a vaginal probe is disclosed for local stimulation of the nerves of the vagina, with heat or cold applied at a temperature change rate of 0.1-20 degree Celsius per second.

During brain surgery there is a general difficulty of determining if tissue about to be damaged serves a crucial brain function. In surgical procedures, patients are typically given a mixture of drugs to have the following three effects: anesthesia (loss of consciousness), pain reduction and immobilization. Due to the difficult in determining if the correct effect has been achieved, there exists a problem of patients which are immobilized but are conscious and/or feel pain during surgery. Even if not conscious, pain can cause an increase in sensed or even chronic pain after surgery.

The following outlines typical diagnostic applications for the nociceptive system:

Quantitative Sensory Testing (QST)

Quantitative Sensory Testing (QST) enables the user to evaluate specific components of the nociceptive system, including pain-mediating thinly myelinated A-delta and unmyelinated C-fibers. QST enables the physician to identify the coexistence of pain with both central and peripheral nervous system abnormalities, aiding to the diagnosis of neuropathic pain syndromes.

Central and Peripheral Nervous System Abnormalities Involving Pain

Small Fiber Neuropathy (SFN)

Small Fiber Neuropathy (SFN) refers to peripheral neuropathies characterized by the impairment of A-delta and C-fibers. SFN is a relatively common disorder resulting in severe and troublesome symptoms (relating to somatic and autonomic nerve fiber impairment), which may be difficult to control (ref—Hoitsma E. et al., “Small fiber neuropathy: a common and important clinical disorder”, J. Neurol. Sci. (2004), 227(1):119-30). Small fiber functions are most commonly investigated by QST devices for the determination of thermal perception and thermal pain thresholds. Recent works have shown that warm and heat-pain threshold correlated with quantification of Intra-Epidermal Nerve Fiber (IENF) density (ref—Laurie G., “Small fiber neuropathies”, Curr. Opin. Neurol. (2005), 18(5):591-7). IENF are somatic unmyelinated C-fibers, which density can be quantified with a skin biopsy. Skin biopsy can demonstrate the loss of IENF in SFN. Although this technique is invasive, it is currently performed in clinics and in universities. Moreover, in the presence of additional underlying medical conditions such as Diabetes, skin biopsy is considered harmful.

Disorders of the Central Nervous System (CNS)

Sensory symptoms are common in diseases of the Central Nervous Systems (CNS) such as stroke, multiple sclerosis and syringomyelia. Even without presence of central pain, sensory symptoms can be disturbing and an impact on patient quality of life. QST can be used for the assessment of patients with CNS dysfunction as a more precise means to quantify sensory loss than the ordinary bedside techniques. Thermal QST can also be used to monitor the functioning of the spinothalamic tract, one of the major ascending somatosensory pathways (ref—Zaslansky R. et al., “Clinical applications of quantitative sensory testing (QST)”, J. Neurol. Sci. (1998), 153(2):215-38).

Spinal Cord Lesions & Radiculopathy

Radiculopathy is primarily caused by herniated disc pressure on the nerve root near the spinal cord. It is common for pain to occur with radiculopathy indicating that small fibers also became irritated, mechanically or chemically. QST can be used to explore the different populations of nerve fibers and dermatomes involved in lumbar Radiculopathy and to evaluate the severity of sensory dysfunction (ref—Nygaard O P. et al., “The function of sensory nerve fibers in lumbar radiculopathy. Use of quantitative sensory testing in the exploration of different populations of nerve fibers and dermatomes”, Spine (1998), 23(3):348-52). Furthermore, thermal QST can predict the degree of small fiber recovery following surgical decompression in the nerve root.

One of the most effective treatment options of the radicular neuropathic pain syndromes is Spinal Cord Stimulation (SCS). QST can be utilized to investigate the long term peripheral effects of SCS on sensation.

QST is also beneficial in discriminating between assessment of preserved sensation and subclinical deficit (ref—Nygaard O P et al., “Recovery of sensory nerve fibers after surgical decompression in lumbar radiculopathy: use of quantitative sensory testing in the exploration of different populations of nerve fibers” J. Neurol. Neurosurg. Psychiatry (1998), 64(1):120-3). Furthermore, QST provides better clinical detection of natural recovery or changes in level of injury following interventions designed to repair spinal cord injuries (SCI, ref—Nicotra A. et al., “Thermal perception thresholds: assessing the level of human spinal cord injury”, Spinal Cord (2006), 44(10):617-24).

Results of QST in whiplash patients may serve as an objective diagnostic tool for the assessment of possible damage to small sensory nerve fibers and damage to the central trigeminal pathway in the upper spinal cord segments. Raised thermal thresholds in patients with chronic symptoms after whiplash injury may also suggest damage to the central trigeminal pathway in the upper spinal cord segments and the ponto-medullary levels of the brainstem (ref-Zaslansky R. et al., “Clinical applications of quantitative sensory testing (QST)”, J. Neurol. Sci. (1998), 153(2):215-38 or Nygaard O P. et al., “The function of sensory nerve fibers in lumbar radiculopathy. Use of quantitative sensory testing in the exploration of different populations of nerve fibers and dermatomes”, Spine (1998), 23(3):348-52).

Diffuse Noxious Inhibitory Control (DNIC)

Diffuse Noxious Inhibitory Control (DNIC, also known as Conditioned Pain Modulation CPM) test paradigm is an advanced physcophysical test for the assessment of efficiency of the Endogenous Analgesia (EA) system. The individual efficiency of the EA system is of high clinical relevance in the characterization of one's capability to modulate pain, and consequently one's susceptibility to pain disorders.

Thermal stimulation devices can be used in the assessment of DNIC efficiency as the conditioned (test) stimulus. As was shown by Yarnitsky's group (ref—Yarnitsky D. et al., “Prediction of chronic post-operative pain: pre-operative DNIC testing identifies patients at risk”, Pain (2008), 138(1):22-8), low DNIC efficiency was associated with higher intensity post-operative pain, indicating that efficiency of DNIC can predict the patient's susceptibility to suffer from chronic post-operative pain. Assessment of the EA system before procedures that might generate pain may allow individually tailored pain prevention and management, which may substantially reduce suffering (ref—Yarnitsky D. et al., “Prediction of chronic post-operative pain: pre-operative DNIC testing identifies patients at risk”, Pain (2008), 138(1):22-8).

Commercially available systems for physiological temperature stimulation were generally constructed for research use. As such, cost, size and ease of use were of secondary importance and the systems were designed to cover large range of stimulation parameters. To achieve such goals, the systems were not optimized for clinical testing and screening use. For example, systems of the art use liquid heat exchangers which are cumbersome, expensive and less reliable.

There is a need, and it will be advantageous to design a system for thermal stimulation of tissue that is optimized for clinical screening and testing. In such a system, the parameters ranges should be narrow, and limited to the parameters used in clinical rather than in research laboratory settings.

### SUMMARY OF THE INVENTION

A thermal sensory analyzer system is provided, with limited parameter ranges which enable simpler, smaller and cheaper construction and operation. The thermal sensory analyzer system is a QST (Quantitative Sensory Testing) device including advanced software package designed for clinical use and advance research in the field of pain management as well as neurology and neurophysiology.

The thermal stimulation probe is a small and compact system, designed especially for the clinical environment. Its design and specification are targeted especially for quantitative assessment of small nerve fiber dysfunctions according to recently established protocols, such as the protocol established by the DFNS (German Research Network on Neuropathic Pain) or other protocols.

The thermal stimulation probe is capable to generate controlled, accurate thermal stimuli. Furthermore, the thermal stimulation probe system enables the user to perform various thermal test paradigms, controlling the temperature and the duration, including the methods of Limits, Levels, TSL (Thermal Sensory Limen), “Ramp and Hold” and more. These test paradigms can be utilized for a wide range of thermal QST pain measures such as thermal detection thresholds, heat or cold induced pain thresholds, tolerance, temporal summation, Diffuse Noxious Inhibitory Control (DNIC, known also as CPM) and others.

The thermal stimulation probe system comprises the following components:

Main (electronic) unit (seen in FIGS. 2A-2H)

Thermode (seen in FIGS. 1A-1C)

Patient response unit (seen in FIG. 3)

Medical-grade power adaptor (not seen in these figures)

USB cable adaptor (not seen in these figures)

Software application—Medoc Main Station (executed for example on a PC, laptop or the likes, a representative screen is seen in FIG. 4)

According to one aspect, a portable device for thermal stimulation of tissue of a patient is provided, the portable device comprising:

a heat control element having a proximal side and a distal side wherein a temperature difference can be generated between said proximal side and said distal side, and wherein said proximal side is capable of contacting the tissue;

a heat sink coupled to said distal side wherein said heat sink is capable of dispersing excess heat resulting from the temperature difference;

a fan capable of exchanging heat with surrounding air;

at least one resilient element connected between said heat sink and said fan, wherein said at least one resilient element is supporting said fan so as to prevent direct contact between said heat sink and said fan;

at least one temperature sensor; and

circuitry that activates said heat control element using said at least one temperature sensor, so as to achieve a desired temperature stimulation of the tissue at a rate of substantially 1 degree Celsius per second.

In some embodiments, the temperature stimulation of the tissue is at a heating rate of 0.1-2 degrees Celsius per second.

In some embodiments, the temperature stimulation of the tissue is at a cooling rate of 0.1-1 degrees Celsius per second.

In some embodiments, the temperature stimulation of the tissue is at a rate sufficiently slow for preventing false triggering of A-delta fibers in the tissue.

In some embodiments, the at least one resilient element further prevents direct contact between the fan and the heat control element.

In some embodiments, the prevented direct contact between the fan and the heat control element may prevent false triggering of fibers in the tissue stimulated by vibrations caused by the fan.

In some embodiments, the portable device further comprises a patient response unit capable of receiving feedback from the patient during stimulation.

In some embodiments, the patient response unit comprises at least one button to be pressed by the patient if stimulated by temperature change at the tissue.

In some embodiments, the heat control element comprises a Peltier element.

In some embodiments, the fan is covered with a perforated case shell.

In some embodiments, the portable device can be used at any space where sufficient electric power may be supplied.

In some embodiments, positioning of the portable device is fixed by mounting it onto a wall.

In some embodiments, the portable device further comprises a medical-grade power adaptor.

In some embodiments, the portable device further comprises a Universal Serial Bus (USB) cable adaptor.

According to another aspect, a method for thermal stimulation of tissue of a patient is provided, the method comprising:

providing a heat control element having a proximal side and a distal side wherein a temperature difference can be generated between said proximal side and said distal side;

coupling said heat control element to at least one temperature sensor;

contacting said proximal side with the tissue;

changing the temperature of said heat control element, relative to a neutral temperature of the tissue at a rate of substantially 1 degree Celsius per second, using said at least one temperature sensor; and

receiving feedback from the patient responding to the stimulation, using a patient response unit.

In some embodiments, the method further comprises:

providing a heat sink coupled to said distal side, and capable of dispersing the excess heat resulting from the temperature changes;

providing a fan capable of exchanging heat with surrounding air; and

providing at least one resilient element connecting between said heat sink and said fan, wherein said at least one resilient element is supporting said fan so as to prevent direct contact between said heat sink and said fan.

In some embodiments, the temperature stimulation of the tissue is at a heating rate of 0.1-2 degrees Celsius per second.

In some embodiments, the temperature stimulation of the tissue is at a cooling rate of 0.1-1 degrees Celsius per second.

In some embodiments, the temperature stimulation of the tissue is at a rate sufficiently slow for preventing false triggering of A-delta fibers in the tissue.

In some embodiments, the at least one resilient element further prevents direct contact between the fan and the heat control element.

In some embodiments, the fan is covered with a perforated case shell.

In some embodiments, the prevented direct contact between the fan and the heat control element may prevent false triggering of fibers in the tissue stimulated by vibrations caused by the fan.

In some embodiments, the patient response unit comprises at least one button to be pressed by the patient if stimulated by temperature change at the tissue.

In some embodiments, the heat control element comprises a Peltier element.

In some embodiments, the method further comprises providing a medical-grade power adaptor.

In some embodiments, the method further comprises providing a Universal Serial Bus (USB) cable adaptor.

In some embodiments, the data gathered from said heat control element with said at least one temperature sensor is displayed on a graphical user interface designed for a clinical environment, and executed on a PC, laptop or a similar device.

In some embodiments, the method further comprises preforming at least one of the following safeguard mechanisms:

a temperature limit test, where heating is stopped when temperature reaches an upper predetermined temperature limit;

a time limit test where heating is stopped when heating exceeds a maximum predetermined time allowed; and

a continuous system test, where heating is halted when a malfunction is detected during system operation.

In some embodiments, the method further comprises preforming at least one of the following safeguard mechanisms:

a temperature limit test, where cooling is stopped when temperature reaches a lower predetermined temperature limit;

a time limit test where cooling is stopped when cooling exceeds a maximum predetermined time allowed; and

a continuous system test, where cooling is halted when a malfunction is detected during system operation.

In some embodiments, the method further comprises preforming a temperature limit test with gradual cooling, when temperature reaches an upper predetermined temperature limit, until a predetermined neutral temperature is reached.

In some embodiments, the method further comprises preforming a temperature limit test with gradual heating, when temperature reaches a lower predetermined temperature limit, until a predetermined neutral temperature is reached.

In some embodiments, the heat control element executes various thermal test paradigms of at least one of the following methods of Limits, Levels, Thermal Sensory Limen (TSL), and Ramp and Hold.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A shows an exploded view of a thermal stimulation probe (thermode), according to an exemplary embodiment.

FIG. 1B shows a view of the thermal stimulation thermode unit, according to an exemplary embodiment.

FIG. 1C shows a top image of the thermal stimulation probe, according to an exemplary embodiment.

FIG. 1D shows a bottom image of the thermal stimulation probe, according to an exemplary embodiment.

FIG. 2A shows an image of the electronics box of the thermal stimulation probe system, according to an exemplary embodiment.

FIG. 2B shows another image of the electronics box of the thermal stimulation probe system, according to an exemplary embodiment.

FIG. 2C shows another image of the electronics box of the thermal stimulation probe system, according to an exemplary embodiment.

FIG. 2D shows yet another image of the electronics box of the thermal stimulation probe system, according to an exemplary embodiment.

FIG. 2E shows a drawing of the electronics box of the thermal stimulation probe system, according to an exemplary embodiment.

FIG. 2F shows another drawing of the electronics box of the thermal stimulation probe system, according to an exemplary embodiment.

FIG. 2G shows another drawing of the electronics box of the thermal stimulation probe system, according to an exemplary embodiment.

FIG. 2H shows yet another drawing of the electronics box of the thermal stimulation probe system, according to an exemplary embodiment.

FIG. 3A shows a patient response unit of the thermal stimulation probe system, according to an exemplary embodiment.

FIG. 3B shows an image of the thermal stimulation probe system, according to an exemplary embodiment.

FIG. 4 shows an exemplary screen of the software used with the thermal stimulation probe system, according to an exemplary embodiment.

FIG. 5 shows a drawing of the calibration masks for the thermal stimulation probe system, according to an exemplary embodiment.

### DESCRIPTION OF THE PREFERRED EMBODIMENTS

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For clarity, non-essential elements were omitted from some of the drawings.

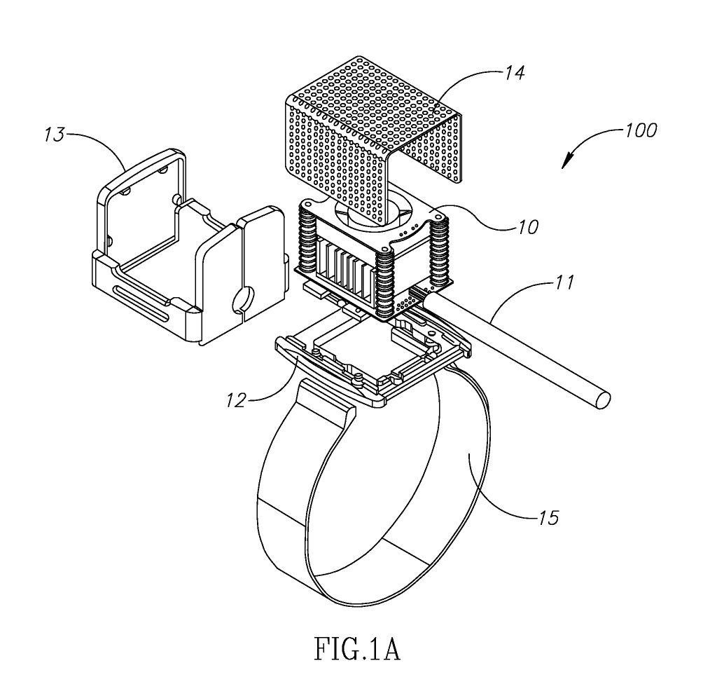

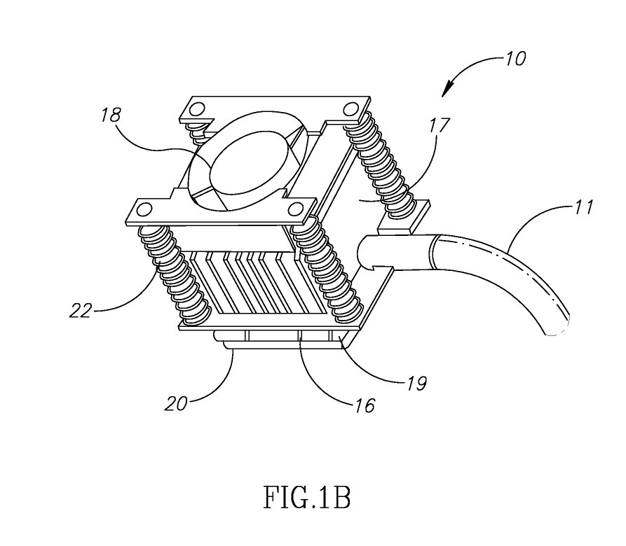

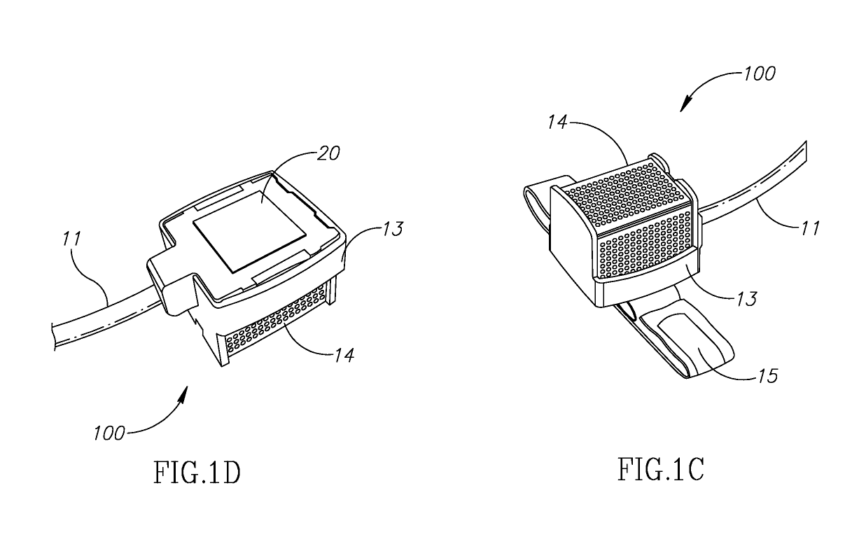

FIG. 1A shows an exploded view of a thermal stimulation probe 100, and FIG. 1B shows a view of the thermode unit 10, according to an exemplary embodiment. The thermal stimulation probe 100 delivers a thermal stimulus to a tested subject and comprises the following main components:

A thermoelectric cooler (TEC 19)

Temperature sensitive resistors (thermistors 16)

Contact plate (20)

Heat exchangers (a heat sink 17, and a fan 18)

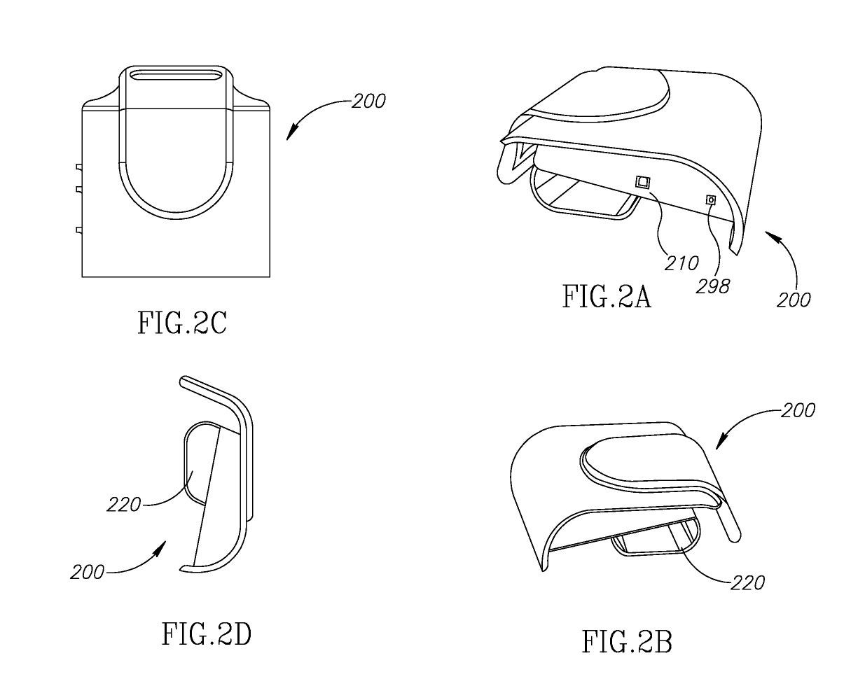

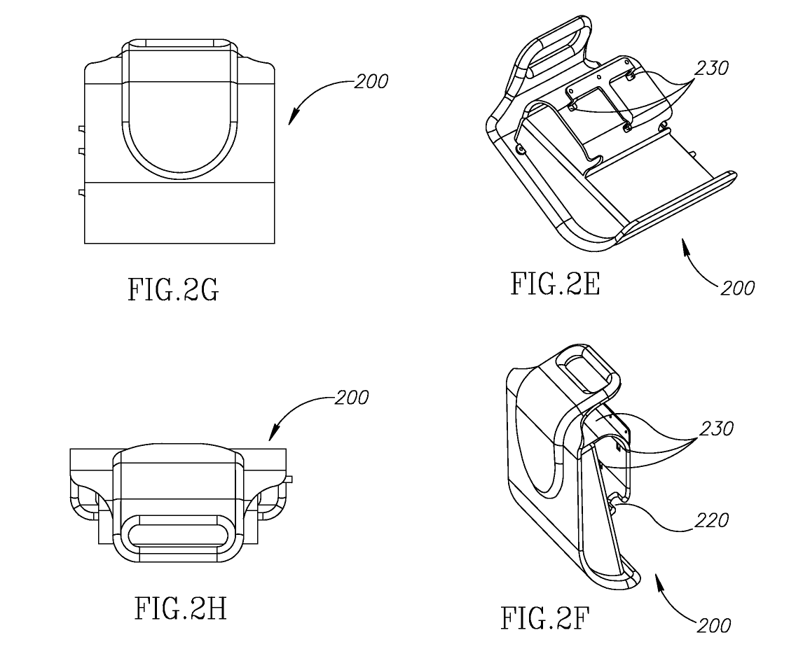

In contrast to thermal probes of the art, for example as seen in FIGS. 1 and 2 of US application 2012/0095535, and FIG. 1 of U.S. Pat. No. 5,191,896, the probe 100 seen in FIGS. 1A and 1B of the current application does not require liquid heat exchange. Instead, a heat sink 17 and fan 18 are used.

Control of temperature changes at the thermode is achieved with circuitry (not seen in the figures), located on a PCB between the heat sink 17 and the TEC 19, using a heat control element of a Peltier element as the thermoelectric cooler 19). The Peltier element generates a temperature difference between its distal and proximal plates which can be controlled by the amount and direction of the current flowing through its poles.

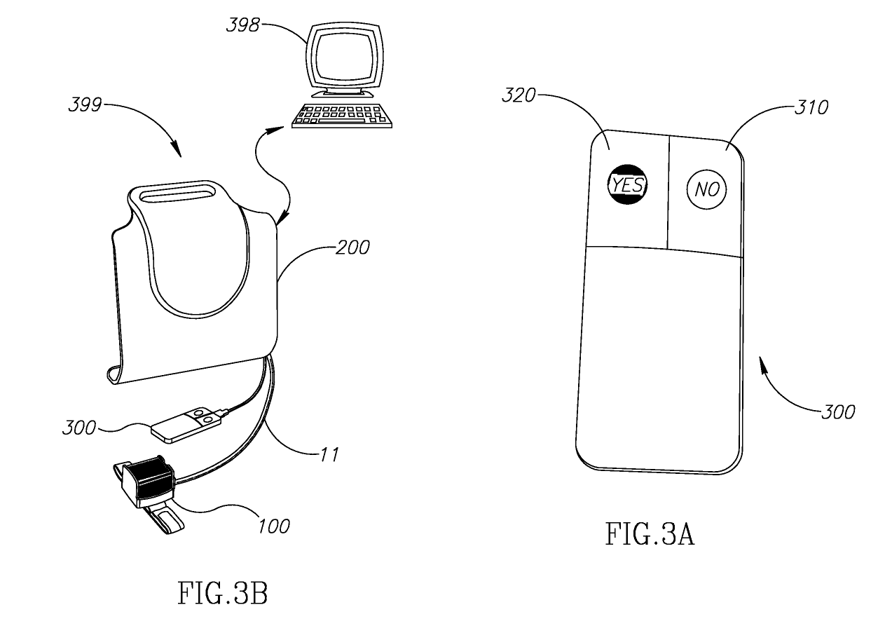

The thermoelectric cooler (TEC) 19 uses a heat sink 17 and fan 18 to directly exchange heat with the surrounding air. Thus, the thin connecting cable 11 linking the thermal head to the control unit (seen in FIG. 3B) does not include liquid pipes. The absence of liquid pipes enables easy connection/disconnection of probes, with more flexible and possibly longer cabling. Furthermore, the absence of liquid pipes also enables having a small and portable structure.

The thermode unit 10 is placed into a base 12 with case shell 13, and covered with a perforated case shell 14 on top. The perforated case shell 14 is placed on top of the fan 18 so as to allow air flow to and from the fan 18. The proximal side of the thermode 10 may be placed in contact to a body part (not shown) and optionally fastened with straps 15, which are connected to the base 12.

The thermode 10 is calibrated to ensure accuracy of measured temperature. The thermal stimulation probe system monitors the thermode 10 temperature in real time at intervals of 5 msec. The temperature of the thermode 10 is controlled via a PID (Proportional Integral Derivative) based algorithm which determines the power supplied to the thermode 10 at any given time, with the required temperature defined according to the operating program. The thermal stimulation device temperature control mechanism ensures that the temperature remains within tolerance of the required temperature.

The TEC 19 is the active element upon which the temperature gradient is generated. The temperature is mediated from the TEC 19 to the external surface of the proximal side of the thermode 10 via the contact plate 20. The thermistors 16 are used as temperature sensors in the circuitry of the temperature control process to measure the current temperature and feed the data directly into the control circuit. Heat exchangers (a fan 18 and heat sink 17) are used to disperse the excess heat resulting from the temperature changes on the TEC 19.

For a smaller portable system, the cooling technology of the thermode 10 implements an air cooling mechanism based on a heat sink 17 mounted directly on the distal side of the Peltier element at TEC 19, and a fan 18. Resilient elements (e.g. springs) 22 hold the frame of the fan 18 above the heat sink 17, without direct contact between the frame of the fan 18 and the heat sink 17 or TEC 19 so that vibrations, caused by the fan's 18 movements and effecting the perceived sensation of a patient, are reduced and thus no additional nerve fibers are stimulated. To further improve safety, the power source may be reduced to 12V.

The temperature range of the thermode 10 is ˜20-50° C., with a heating rate of ˜0.1-2° C. per second and a cooling rate of ˜0.1-1° C. per second. In order to allow the thermode to cool down back to a neutral temperature in a safe manner, an operation interval of several minutes may be taken between different patients.

Accordingly, the combination of the slow heating rate and/or cooling rate with the reduction of the fan's 18 vibrations enables measurements with high accuracy (˜0.1° C. per second), and thus improving the stimulation process due to the following reasons:
resolution of pain thresholds is in the order of ˜1° C., so that absolute measurement accuracy contributes to reliable and repetitive results.
slow stimulation activates unmyelinated C-fibers, and prevents false triggering of the myelinated A-delta fibers (which respond to fast and sharp pain).
maintaining identical accuracy in the measurements between different tests, and also between different stimulating devices, may contribute to having accurate repetitive and robust measurements.
in the test paradigm method of “Limits” the response time of the patient (not simultaneous with the sensing of pain, where the temperature keeps changing until the patient responds) is a bias factor, so that a controlled and slow rate contributes to reducing the effect of threshold response time.
vibrations mostly stimulate a different kind of nerve fibers (A-alpha and A-beta) and may also effect the stimulation of the A-delta fibers and thus change the entire measurement. In order not to mix these kinds of stimulations, and to isolate and measure the proper responding fibers, the vibrations are reduced.

FIGS. 1C and 1D show top and bottom images of the thermal stimulation probe 100 respectively, according to an exemplary embodiment. The compact construction of the probe 100, with contact plate 20, thin connecting cable 11, case shell 13, and air venting openings in the perforated case shell 14 are clearly seen.

FIGS. 2A-2D show images of the electronics box 200 of the thermal stimulation probe system, according to an exemplary embodiment. FIGS. 2A and 2B show isometric views where the electronics box 200 is placed on a surface. In FIG. 2A a power input port 298 and a data (USB) port 210 can be seen. In FIG. 2B a probe placement slot 220 can be seen at the back side of the electronics box 200. FIG. 2C shows a front view, while FIG. 2D shows a side view of the electronics box 200.

FIGS. 2E-2H show drawings of the electronics box 200 of the thermal stimulation probe system, according to an exemplary embodiment. FIG. 2E shows isometric back view, while FIG. 2F is an isometric side view. In FIGS. 2E and 2F a wall hanging structure 230 and a probe placement slot 220 (for placing a probe such as the probe 100 seen in FIGS. 1A-1C) can be seen. The electronics box 200 of the thermal stimulation probe system may be fixed to a wall with the wall hanging structure 230 or may be completely portable and placed near the patient. FIGS. 2G and 2H show front and top view drawings respectively, of the electronics box 200.

FIG. 3A shows a patient response unit 300 of the thermal stimulation probe system, according to an exemplary embodiment. The patient response unit 300 is used by the patient to indicate by pressing the “YES” 320 or “NO” 310 buttons depending if he/she can or cannot feel the stimulation applied by the probe 100 during the examination.

FIG. 3B shows an image of the whole thermal stimulation probe system 399, according to an exemplary embodiment. The thermal stimulation probe system 399 comprises: The thermal stimulation probe 100 and patient response unit 300 which are connected via cables to the electronics box 200 of the thermal stimulation probe system, and a computer 398 connected to electronics box 200.

Upon start-up the system 399 performs a self-test in which system sensors, active elements and safety shut-down are being tested. If a malfunction is detected, an appropriate message is displayed and the system 399 cannot operate until that malfunction is resolved.

Several safeguard mechanisms are implemented in the system 399 to safeguard against extreme temperatures and to protect the tested subject as well as the unit. Safeguard mechanisms comprise of both software based protection and hardware based protection.

The software protection may include one or several of the following test options:
Temperature limit—heating may stop when the thermode temperature reaches the upper predetermined temperature limit. Alternatively, cooling may stop when the thermode temperature reaches the lower predetermined temperature limit. Temperature vs. time limit—heating or cooling may stop when the thermode temperature exceeds the maximum predetermined time duration allowed.
Continuous system tests—sensor functions are monitored during system operation. In case any malfunction is detected in the thermode, heating or active cooling is immediately halted.
Temperature control integrity—the integrity of PID temperature control is monitored during system operation. In case any malfunction is detected, power to the thermodes is immediately disabled.
   The hardware protection overrides any software control and disconnects power to the thermode 10 if the temperature exceeds 57° C., with additional protection on the heat sink 17 temperature. Additionally, the hardware protection may only indicate when the temperature exceeds 57° C. so that the software will control the thermode 10 cooling until a predetermined temperature (e.g. 30° C.) may be reached in a gradual and controlled rate. Temperature limits and time limits may be defined according the safety standards provided by the FDA.

The system automatically detects if a thermode 10 has been disconnected, and disables power to it in order to protect both system and user. Additionally, the integrity of communication between the computer 398 and the thermal stimulation system 100 is monitored, where the power supply to the thermode 10 is disconnected in case of communication loss.

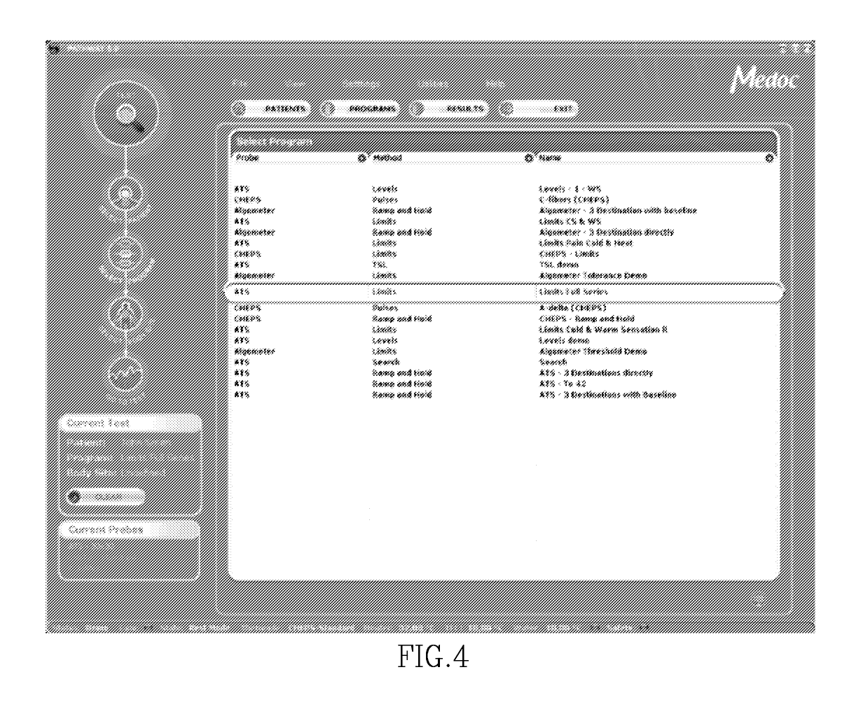

FIG. 4 shows an exemplary screen of the software used with the thermal stimulation probe system. The software is executed and displayed on a graphical user interface (e.g. computer 398). The software contains SQL based data base to allow complete patient, program and results management with programmable parameters such as “Adaptation Temperature”, “Heating Rate”, “Cooling Rate”, “Number of Stimuli”, “Sound Option”, and “Randomize Option”. The user friendly interface allows easy test management and may provide real-time visual and auditory stimulation feedback, providing a full report at the end of the test. Results may be displayed in color customizable reports or exported to MS Excel for further analysis. Management and customization of body site and normative data is available as well, as the test operation may change according to different selected body sites.

The thermal stimulation probe may be utilized as a standalone unit and may also connect with a “Medoc AlgoMed” algometer (available from Medoc Ltd., Ramat Yishai, Israel and seen for example in http://www.medoc-web.com/products/). The thermal stimulation probe system provides pain diagnostic testing with digital clarity and computer interface for data logging. Thermal QST is a reliable measure of pain in pain management practice. The thermal stimulation probe thus may prove the benefits of applied medication, physiotherapy or manipulation. Additional devices may operate with the thermal stimulation probe (e.g. a continuous VAS evaluation unit) using a standard Universal Serial Bus (USB) connection to the computer 398.

As treatment progresses, the thermal stimulation probe system quantifies improvements or setbacks. Accordingly, with pain threshold measurements providing information not obtainable by any other method, the quantitative measurements may give reassurance to patients by confirming improvement.

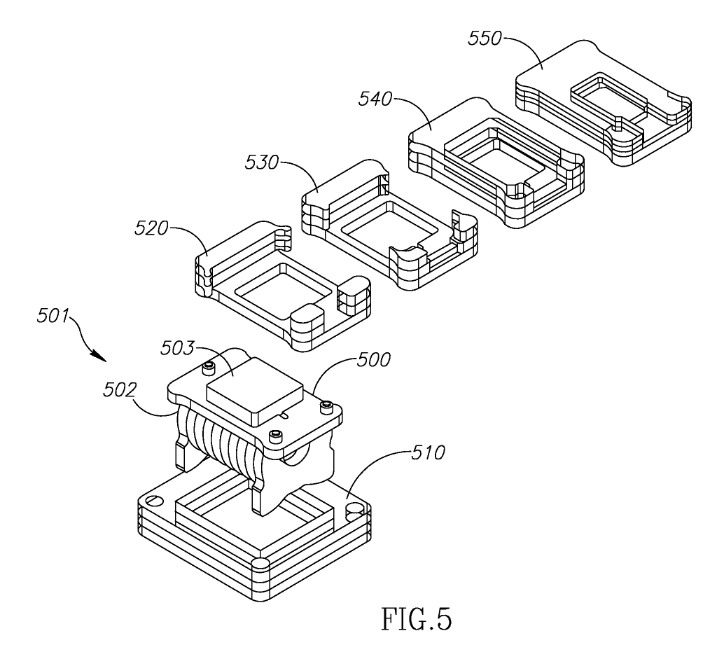

FIG. 5 shows a drawing of a calibration device 501 for the thermal stimulation probe system, according to an exemplary embodiment. This calibration device 501 is suitable for use with the current thermal stimulation probe system as well as with other systems available from Medoc Ltd. (e.g. Pathway and TSA-II), where in a single device different masks 520, 530, 540 and 550 are used for different kinds of thermodes, replacing the need for multiple calibration systems. A main body 500, having a cylindrically shaped bottom part 502 and a temperature sensor on the top part 503, is fitted with a sponge (not seen in FIG. 5) which may be smeared with thermal grease in order to improve thermal conduction. In operation, a selection of calibration masks 520, 530, 540 and 550 (suitable for the thermode to be calibrated, e.g. mask 530 for the current thermal stimulation probe system) may be fitted onto the top part 503 of the main body 500 with the thermode placed onto the mask 520, 530, 540 and 550. Straps attached to the thermode may be used to fasten the thermode to the cylindrically shaped bottom part 502 of the main body 500, for a stable contact with the temperature sensor. Finally the main body 500 with the thermode may be fitted onto the base unit 510, where the base unit 510 may be fixed to any platform in order to further stabilize the main body 510. By connecting the thermode to an external thermometer the thermode may be calibrated with suitable software executed on an external computer.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A portable device for thermal stimulation of tissue of a patient comprising:
a heat control element having a proximal side and a distal side wherein a temperature difference can be generated between said proximal side and said distal side, and wherein said proximal side is capable of contacting the tissue;
a heat sink coupled to said distal side wherein said heat sink is capable of dispersing excess heat resulting from the temperature difference;
a fan capable of exchanging heat with surrounding air;
at least one resilient element connected between said heat sink and said fan, wherein said at least one resilient element is supporting said fan so as to prevent direct contact between said heat sink and said fan;
at least one temperature sensor; and
circuitry that activates said heat control element using said at least one temperature sensor, so as to achieve a desired temperature stimulation of the tissue at a rate of substantially 1 degree Celsius per second.

2.-4. (canceled)

5. The portable device according to claim 1, wherein the at least one resilient element further prevents direct contact between the fan and the heat control element.

6. The portable device according to claim 2, wherein the prevented direct contact between the fan and the heat control element prevents false triggering of fibers in the tissue stimulated by vibrations caused by the fan.

7. The portable device according to claim 1, further comprising a patient response unit capable of receiving feedback from the patient during stimulation.

8. The portable device according to claim 4, wherein the patient response unit comprises at least one button to be pressed by the patient if stimulated by temperature change at the tissue.

9. The portable device according to claim 1, wherein the heat control element comprises a Peltier element.

10. The portable device according to claim 1, wherein the fan is covered with a perforated case shell.

11-14. (canceled)

15. A method for thermal stimulation of tissue of a patient, comprising:
providing a heat control element having a proximal side and a distal side wherein a temperature difference can be generated between said proximal side and said distal side;
providing a heat sink coupled to said distal side, and capable of dispersing the excess heat resulting from the temperature changes;
providing a fan capable of exchanging heat with surrounding air;
providing at least one resilient element connecting between said heat sink and said fan;
coupling said heat control element to at least one temperature sensor;
contacting said proximal side with the tissue;
changing the temperature of said heat control element, relative to a neutral temperature of the tissue at a rate of substantially 1 degree Celsius per second, using said at least one temperature sensor; and
receiving feedback from the patient responding to the stimulation using a patient response unit,
wherein said at least one resilient element is supporting said fan so as to prevent direct contact between said heat sink and said fan.

16. (canceled)

17. The method according to claim 8, wherein the temperature stimulation of the tissue is at a heating rate of 0.1-2 degrees Celsius per second.

18. The method according to claim 8, wherein the temperature stimulation of the tissue is at a cooling rate of 0.1-1 degrees Celsius per second.

19. The method according to claim 8, wherein the temperature stimulation of the tissue is at a rate sufficiently slow for preventing false triggering of A-delta fibers in the tissue.

20. The method according to claim 8, wherein the at least one resilient element further prevents direct contact between the fan and the heat control element.

21. The method according to claim 12, wherein the prevented direct contact between the fan and the heat control element prevents false triggering of fibers in the tissue stimulated by vibrations caused by the fan.

22. (canceled)

23. The method according to claim 8, wherein the patient response unit comprises at least one button to be pressed by the patient if stimulated by temperature change at the tissue.

24-26. (canceled)

27. The method according to claim 8, wherein data gathered from said heat control element with said at least one temperature sensor is displayed on a graphical user interface designed for a clinical environment, and executed on a PC, laptop or a similar device.

28. The method according to claim 8, wherein the method further comprises preforming at least one of the following safeguard mechanisms:
a temperature limit test, where heating is stopped when temperature reaches an upper predetermined temperature limit;
a time limit test where heating is stopped when heating exceeds a maximum predetermined time allowed; and
a continuous system test, where heating is halted when a malfunction is detected during system operation.

29. The method according to claim 8, wherein the method further comprises preforming at least one of the following safeguard mechanisms:
a temperature limit test, where cooling is stopped when temperature reaches a lower predetermined temperature limit;
a time limit test where cooling is stopped when cooling exceeds a maximum predetermined time allowed; and
a continuous system test, where cooling is halted when a malfunction is detected during system operation.

30. The method according to claim 16, wherein the method further comprises preforming a temperature limit test with gradual cooling, when temperature reaches an upper predetermined temperature limit, until a predetermined neutral temperature is reached.

31. The method according to claim 17, wherein the method further comprises preforming a temperature limit test with gradual heating, when temperature reaches a lower predetermined temperature limit, until a predetermined neutral temperature is reached.

32. The method according to claim 8, wherein the heat control element executes various thermal test paradigms of at least one of the following methods of Limits, Levels, Thermal Sensory Limen (TSL), and Ramp and Hold.
